(19) United States
(12) Patent Application Publication
(10) Pub. No.: US 2026/0315649 A1
(43) Pub. Date: Oct. 15, 2026

(54) FLEXIBLE, TRANSPARENT ORGANOHYDROGEL FOR SKIN-BASED APPLICATIONS

(71) Applicant: THE REGENTS OF THE UNIVERSITY OF MICHIGAN, Ann Arbor, MI (US)

(72) Inventors: Lingjie Jay GUO, Ann Arbor, MI (US); Jennie Jean PAIK, Ann Arbor, MI (US); Boonjae JANG, Ann Arbor, MI (US)

(73) Assignee: THE REGENTS OF THE UNIVERSITY OF MICHIGAN, Ann Arbor, MI (US)

(21) Appl. No.: 19/483,692

(22) PCT Filed: May 13, 2024
(86) PCT No.: PCT/US2024/029050

§ 371 Date: Nov. 12, 2025

Related U.S. Application Data
(60) Provisional application No. 63/466,026, filed on May 12, 2023.

(51) Int. Cl. A61L 26/00; A61L 31/04; A61L 31/12; A61L 31/14; A61L 31/16

(52) U.S. Cl. CPC A61L 26/0095; A61L 26/0014; A61L 26/0066; A61L 26/008; A61L 31/048; A61L 31/128; A61L 31/145; A61L 31/16; A61L 2300/216; A61L 2300/406

(57) ABSTRACT

A flexible, transparent organohydrogel for skin-based applications includes a polymeric matrix that includes crosslinked hydrophilic polymers, a divalent metal salt configured to disrupt crystalline interactions of the polymeric matrix, and a polyol configured to reduce a strength of Coulomb attraction among cations and anions of the divalent metal salts and among polymer chains of the polymeric matrix thereby increasing the flexibility of the organohydrogel.

### CROSS-REFERENCE TO RELATED APPLICATIONS

This application claims the benefit of U.S. Provisional Application No. 63/466,026, filed on May 12, 2023. The entire disclosure of the above application is incorporated herein by reference.

### FIELD

The present disclosure relates to flexible, transparent organohydrogel for skin-based applications.

### BACKGROUND

This section provides background information related to the present disclosure which is not necessarily prior art.

The rising demand for cost-efficient and user-friendly wearable electronic devices has led to the emergence of stretchable electronics. However, current wearable devices often utilize stiff sensors that can perform poorly due to the non-conformal contact with the soft, curvilinear surface of the human or animal skin. Next-generation wearable devices must be conformal and maintain electrical connectivity under duress to avoid negatively impacting sensing performance. While many flexible electronic devices are fabricated on polymeric substrates (such as poly(ethylene terephthalate) (PET), polydimethyl siloxane (PDMS), or polyimides (PI)), these substrates are not suitable for stretchable electronics due to their rigidity and low dynamic range. The commercialization of next-generation wearable devices requires biocompatibility, cost-efficiency, and aesthetics in addition to performance. Accordingly, there is a need for a nontoxic, durable, and functional soft material for biointerface applications.

There is also a rising demand for skin care products designed to improve quality of life and comfort, including those directed to wound healing and scar prevention, such as after plastic surgery. There is a need for nontoxic, durable, and functional soft material that can be used for wound healing and scar prevention and also to prevent, treat, or diagnosis other medical conditions.

Poly(vinyl alcohol) hydro- and organogel based materials are a promising material for biointerface applications due to their nontoxicity, cost-effectiveness, and biocompatibility. Flexible hydrogel strain sensors based on poly(vinyl alcohol)—an FDA approved polymer material of use on or as implants in the human body—have gained significant interest in recent years, with a heavy focus on conductivity, stretchability, and mechanical toughness. Previous strategies for achieving conductivity of the poly(vinyl alcohol) hydro- and organogel based materials have involved doping the poly(vinyl alcohol) hydro- and organogel based materials with salts (such as sodium chloride, zinc sulfate, sodium borate, iron (III) chloride, calcium chloride, and/or lithium chloride). Higher gauge factors and wider dynamic range have been achieved by using conductive polymers like poly(3,4-ethylenedioxythiophene) (PEDOT): polystyrene sulfonate (PSS) and/or conductive fillers like graphene, silver nanowires (AgNWs), and/or carbon nanotubes (CNTs). While high-performing, these electrically conducting additives can often be detrimental to biocompatibility, flexibility, and aesthetics of the poly(vinyl alcohol) hydro- and organogel based materials. Further, poly(vinyl alcohol) networks are often formed by repeatedly freezing and thawing an aqueous poly(vinyl alcohol) solution, which results in amorphous domains interspersed with crystalline domains that act as crosslinking points in the network. These crystalline domains can render the gel opaque and impede their ability to dissipate energy under external strain, which is critical to achieve high elongation and high dynamic range. Accordingly, there remains a need for a single nontoxic, durable, and functional soft material for biointerface applications that is transparent, colorless, and comfortable.

### SUMMARY

This section provides a general summary of the disclosure and is not a comprehensive disclosure of its full scope or all of its features.

In various aspects, the present disclosure provides a flexible, transparent organohydrogel for skin-based applications. The flexible, transparent organohydrogel may include a polymeric matrix that includes one or more crosslinked hydrophilic polymers, a divalent metal salt configured to disrupt crystalline interactions of the polymeric matrix, and a polyol configured to reduce a strength of Coulomb attraction among cations and anions of the divalent metal salt(s) and among polymer chains of the polymeric matrix thereby increasing the flexibility of the organohydrogel.

In one aspect, the divalent metal salt may be selected from the group consisting of: magnesium chloride (MgCl₂), zinc chloride (ZnCl₂), zinc nitrate (Zn(NO₃)₂), magnesium nitrate (Mg(NO₃)₂) and calcium chloride (CaCl₂)), and combinations thereof.

In one aspect, the polymeric matrix may include poly(vinyl alcohol) (PVA).

In one aspect, the divalent metal salt may disrupt the crystalline interactions of the polymer matrix may be represented by nOH(PVA):nM²⁺ that occurs in an amount greater than or equal to about 10:1 to less than or equal to about 10:3, where M²⁺ represents the metal group of the divalent metal salt.

In one aspect, the polyol may have a dielectric constant greater than or equal to about 31.7 to less than or equal to about 42.

In one aspect, the flexible, transparent organohydrogel may include greater than or equal to about 5 wt. % to less than or equal to about 60 wt. % of the polyol.

In one aspect, the polyol may include a water-soluble polyol.

In one aspect, the polyol may include glycerol.

In one aspect, the flexible, transparent organohydrogel may have a gauge factor (GF) greater than or equal to about 0.54 to less than or equal to about 1.254.

In one aspect, the flexible, transparent organohydrogel may have a dynamic range greater than or equal to about 5% to less than or equal to about 300%.

In one aspect, the flexible, transparent organohydrogel may have a Young's modulus greater than or equal to about 0.1 kPa to less than or equal to about 7 kPa.

In one aspect, the flexible, transparent organohydrogel may have an ionic conductivity greater than or equal to about 1.79 E⁻⁴ S·cm⁻¹ to less than or equal to about 3.54 E⁻⁴ S·cm⁻¹.

In one aspect, the flexible, transparent organohydrogel may further include a polyphenol.

In one aspect, the polyphenol may be an antibacterial material.

In one aspect, the polyphenol may be selected from the group consisting of: dopamine, tannic acid, gallic acid, and combinations thereof.

In one aspect, the flexible, transparent organohydrogel may include greater than or equal to about 0.01 g to less than or equal to about 0.1 g of the polyphenol for every gram of the crosslinked hydrophilic polymers in the polymeric matrix.

In one aspect, the polymeric matrix may further include hydrophobic molecules configured to improve adhesion for the skin-based applications.

In one aspect, the hydrophobic molecules may include a propyl group.

In various aspects, the present disclosure provides a flexible, transparent organohydrogel for skin-based applications. The flexible, transparent organohydrogel may include a polymeric matrix including poly(vinyl alcohol) (PVA), a divalent metal salt configured to disrupt crystalline interactions of the polymeric matrix, and greater than or equal to about 5 wt. % to less than or equal to about 60 wt. % of glycerol configured to reduce a strength of Coulomb attraction among cations and anions of the divalent metal salts and among polymer chains of the polymeric matrix thereby increasing the flexibility of the organohydrogel. The divalent metal salt may be selected from the group consisting of: magnesium chloride (MgCl₂), zinc chloride (ZnCl₂), zinc nitrate (Zn(NO₃)₂), magnesium nitrate (Mg(NO₃)₂) and calcium chloride (CaCl₂)), and combinations thereof. A ratio of hydroxyl (—OH) groups in the poly(vinyl alcohol) to a metal group of the divalent metal salt may be represented by nOH(PVA):nM²⁺ and may be greater than or equal to about 10:1 to less than or equal to about 10:3.

In one aspect, the flexible, transparent organohydrogel may further include at least one of: a polyphenol having antibacterial properties and a propyl group selected to improve adhesion for the skin-based applications.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1A is a scanning electron microscope image (scale: 20 micrometers) of a surface of an example flexible, transparent organohydrogel prepared in accordance with various aspects of the present disclosure;

FIG. 1B is a scanning electron microscope image (scale: 20 micrometers) of a surface of a poly(vinyl alcohol) hydrogel;

FIG. 1C is a scanning electron microscope image (scale: 20 micrometers) of a surface of a poly(vinyl alcohol)-zinc hydrogel;

FIG. 2A is a graphical demonstration illustrating an increase in resistance response to strains between 10% and 300% for an example flexible, transparent organohydrogel prepared in accordance with various aspects of the present disclosure;

FIG. 2B is a graphical demonstration illustrating a percent resistance change (ΔR/R₀) plotted against strain (%), where the slopes represent a gauge factor (GF) for example for flexible, transparent organohydrogels prepared in accordance with various aspects of the present disclosure;

FIG. 2C is a graphical demonstration illustrating the resistance response to strains for an example of a flexible, transparent organohydrogel in accordance with various aspects of the present disclosure as placed over the hyoid cartilage of a human subject and after a repeated response was elicited. The inset is a photograph of the experimental setup;

FIG. 2D is another graphical demonstration illustrating the resistance response to strains for the example flexible, transparent organohydrogel introduced in FIG. 2C;

FIG. 2E is another graphical demonstration illustrating the resistance response to strains for the example flexible, transparent organohydrogel introduced in FIG. 2C;

FIG. 2F is a graphical demonstration illustrating ionic conductivities characterized using electrical impedance spectroscopy of example flexible, transparent organohydrogels prepared in accordance with various aspects of the present disclosure;

FIG. 3A is a scanning electron microscope image (scale: 20 micrometers) of a surface of an example flexible, transparent organohydrogel prepared in accordance with various aspects of the present disclosure;

FIG. 3B is a scanning electron microscope image (scale: 20 micrometers) of a surface of an example flexible, transparent organohydrogel prepared in accordance with various aspects of the present disclosure;

FIG. 3C is a microscopic image (scale: 20 micrometers) of a surface of an example flexible, transparent organohydrogel prepared in accordance with various aspects of the present disclosure;

FIG. 4A is a graphical demonstration illustrating stress-stain curves of example flexible, transparent organohydrogels prepared in accordance with various aspects of the present disclosure;

FIG. 4B is a graphical demonstration illustrating the Young's modulus and toughness of example flexible, transparent organohydrogels prepared in accordance with various aspects of the present disclosure;

FIG. 4C is a graphical demonstration illustrating energy dissipating properties of example flexible, transparent organohydrogels prepared in accordance with various aspects of the present disclosure;

FIG. 4D is a graphical demonstration illustrating pull-off adhesion properties of example flexible, transparent organohydrogels prepared in accordance with various aspects of the present disclosure;

FIG. 5A is a graphical demonstrating illustrating the Fourier-transform infrared spectroscopy (FTIR) spectra of example flexible, transparent organohydrogels prepared in accordance with various aspects of the present disclosure, where the inset shows possible intermolecular interactions within the example organohydrogels;

FIG. 5B is a graphical demonstrating illustrating the x-ray diffraction (XRD) of example flexible, transparent organohydrogels prepared in accordance with various aspects of the present disclosure, where the insets show proposed models of how polyol loading affects the macromolecular behavior in the organohydrogels;

FIG. 6A are microscopic images demonstrating the antibacterial and non-cytotoxic properties of example flexible, transparent organohydrogels prepared in accordance with various aspects of the present disclosure;

FIG. 6B is a graphical demonstrating illustrating the antibacterial activity of example flexible, transparent organohydrogels prepared in accordance with various aspects of the present disclosure against Staphylococcus aureus, a common gram-positive skin bacterium; and

FIG. 6C is a graphical demonstrating illustrating the antibacterial activity of example flexible, transparent organohydrogels prepared in accordance with various aspects of the present disclosure against Staphylococcus epidermidis, another common gram-positive skin bacteria.

Corresponding reference numerals indicate corresponding parts throughout the several views of the drawings.

### DETAILED DESCRIPTION

Example embodiments are provided so that this disclosure will be thorough and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

When a component, element, or layer is referred to as being “on,” “engaged to,” “connected to,” or “coupled to” another element or layer, it may be directly on, engaged, connected, or coupled to the other component, element, or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer, or section. Terms such as “first,” “second,” and other numerical terms when used herein do not imply a sequence or order unless clearly indicated by the context. Thus, a first step, element, component, region, layer, or section discussed below could be termed a second step, element, component, region, layer, or section without departing from the teachings of the example embodiments.

Spatially or temporally relative terms, such as “before,” “after,” “inner,” “outer,” “beneath,” “below,” “lower,” “above,” “upper,” and the like, may be used herein for ease of description to describe one element or feature's relationship to another element(s) or feature(s) as illustrated in the figures. Spatially or temporally relative terms may be intended to encompass different orientations of the device or system in use or operation in addition to the orientation depicted in the figures.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%.

In addition, disclosure of ranges includes disclosure of all values and further divided ranges within the entire range, including endpoints and sub-ranges given for the ranges.

Example embodiments will now be described more fully with reference to the accompanying drawings.

The rising demand for cost-efficient and user-friendly wearable electronic devices has birthed the field of stretchable electronics that are cost-effective while maintaining sustained adhesion and electrical performance under duress. The present disclosure provides a flexible and/or stretchable, transparent organohydrogel for human or animal skin-based applications. Organohydrogels are water-swollen networks of hydrophilic polymers crosslinked by physical or chemical bonds with structural and mechanical similarities to skin (human and animal) make them good candidates for biointerface applications. The crosslinked, hydrophilic polymers form a soft, transparent matrix that defines the organohydrogel. In various aspects, the hydrophilic polymers include biocompatible, non-acrylate polymers, such as poly(vinyl alcohol) (PVA). In various aspects, the organohydrogels may also include a divalent metal salt and a biocompatible polyol. In still further aspects, the organohydrogels may also include a polyphenol and/or an alkyl group, such as a propyl group, which may be a propyl pendant group or chain. Biocompatible as used herein as used herein refers to non-toxic to cells and tissue of living organisms of the human or animal subject. A “biointerface” as used herein refers to a region of contact between a biomolecule (e.g., cell, biological tissue, living organism, or organic material considered to be living) with another biomaterial or inorganic/organic material.

The terms “flexible” and “stretchable” are meant to refer to the ability of a the organohydrogel to be deformed (for example, into a curved shape) without undergoing a permanent transformation that introduces significant strain, such as strain indicating a failure point of a material, structure, or component. The flexible, transparent organohydrogel may be capable of significant elongation, flexing, bending or other deformation along one or more axes. In certain aspects, the flexible, transparent organohydrogel may undergo a maximum tensile strain of at least about 15% without fracturing; optionally greater than or equal to about 50% without fracturing, optionally greater than or equal to about 75% without fracturing, optionally greater than or equal to about 100% without fracturing, optionally greater than or equal to about 200% without fracturing, optionally greater than or equal to about 300% without fracturing, optionally greater than or equal to about 400% without fracturing, and in certain embodiments, greater than or equal to about 450% without fracturing as will be described in more detail below.

The term “transparent” is meant to refer to the ability of the organohydrogel to be transmissive for a target range of wavelengths of electromagnetic energy, for example, in the visible and ultraviolet wavelength ranges. That is, in certain variations, the flexible, transparent organohydrogel may transmit greater than or equal to about 60% of electromagnetic energy at the predetermined range of wavelengths, optionally of greater than or equal to about 65%, optionally greater than or equal to about 70%, optionally greater than or equal to about 75%, optionally greater than or equal to about 80%, optionally greater than or equal to about 85%, optionally greater than or equal to about 90%, and in certain preferred aspects, optionally greater than or equal to about 95% of the electromagnetic energy at the predetermined range of wavelengths (e.g., in the visible and/or ultraviolet ranges of the spectrum) is transmitted.

In various aspects, the biocompatible, non-acrylate polymer may include a blend of polymers having different molecular weights. For example, the biocompatible, non-acrylate polymer may include polymers (e.g., poly(vinyl alcohol)) having molecular weights greater than or equal to about 29 kDa to less than or equal to about 124 kDa. In certain aspects, the biocompatible, non-acrylate polymer may include a first biocompatible, non-acrylate polymer (e.g., poly(vinyl alcohol)) having a first molecular weight of about 89 kDa and a second biocompatible, non-acrylate polymer (e.g., poly(vinyl alcohol)) having a second molecular weight of about 29 kDa. A ratio of the first biocompatible, non-acrylate polymer to the second biocompatible, non-acrylate polymer may be about 7:3. In other aspects, the biocompatible, non-acrylate polymer may include a first biocompatible, non-acrylate polymer (e.g., poly(vinyl alcohol)) having a first molecular weight of about 89 kDa and a second biocompatible, non-acrylate polymer (e.g., poly(vinyl alcohol)) having a second molecular weight of about 124 kDa. A ratio of the first biocompatible, non-acrylate polymer to the second biocompatible, non-acrylate polymer may be about 7:3. In still other aspects, the biocompatible, non-acrylate polymer may include a first biocompatible, non-acrylate polymer (e.g., poly(vinyl alcohol)) having a first molecular weight of about 29 kDa and a biocompatible, non-acrylate polymer (e.g., poly(vinyl alcohol)) having a second molecular weight of about 124 kDa. A ratio of the first biocompatible, non-acrylate polymer to the second biocompatible, non-acrylate polymer may be about 5:4. Adjusting the ratio of molecular weights may help to tune the tack and rheological properties of the organohydrogel. Skin is naturally hydrophobic. Accordingly, it may be helpful in certain variations to add hydrophobic molecules, like alkyl groups, such as propyl chains, to the polymeric matrix to help to improve the adhesion of the organohydrogel to the skin.

The divalent salt(s) will necessarily have at least one cation and anion, as will be described further below. The divalent metal salt may be selected to disrupt the crystalline matrix interactions of organohydrogel, helping to form a densified amorphous structure that includes, for example, randomly oriented fibrils. The divalent metal salts may help to induce transparency and stretchability by interrupting the formation of the organohydrogel crystallites and thereby modulating mechanical properties. In certain variations, the divalent metal salt may be selected so as to be capable of interacting with the dipole side group on the polymer. When the biocompatible, non-acrylate polymer includes poly(vinyl alcohol) (PVA), the disruption may be represented by nOH(PVA):nM^{2+ and may be greater than or equal to about} 10:1 to less than or equal to about 10:3. In certain variations, the divalent metal salt may include, for example, magnesium chloride (MgCl₂), zinc chloride (ZnCl₂), zinc nitrate (Zn(NO₃)₂), magnesium nitrate (Mg(NO₃)₂), calcium chloride (CaCl₂)), and/or the like. In further variations, the divalent metal salt may be selected from the group consisting of: MgCl₂, ZnCl₂, Zn(NO₃)₂, Mg(NO₃)₂, CaCl₂), and combinations thereof.

The polyol may have a comparatively high dielectric constant so as to effectively reduce the strength of Coulomb attraction amongst cations and anions of the divalent metal salts and amongst polymer chains thereby increasing the flexibility of the organohydrogel. Thus, the polyol provides the organohydrogel with good sensitivity and also high dynamic range by increasing polymer chain mobility and providing more conductive paths for ions to travel. For example, the organohydrogel may have a gauge factor (GF) greater than or equal to about 0.54 to less than or equal to about 1.254, greater than or equal to about 0.84 to less than or equal to about 0.89, optionally about 0.84, and in certain aspects, optionally about 0.89, and a dynamic range greater than 0% to less than or equal to about 400%, optionally greater than or equal to about 5% to less than or equal to about 400%, and in certain aspects, optionally greater than or equal to about 5% to less than or equal to about 300%. In certain aspects, however, high levels of the polyol may increase the Young's modulus, while also reducing the conductivity and sensitivity of the organohydrogel. Accordingly, it is necessary to balance the amounts of the polyol. The organohydrogel preferably has a Young's modulus greater than or equal to about 0.1 kPa to less than or equal to about 7 kPa and an ionic conductivity greater than or equal to about 1.79 E⁻⁴ S·cm⁻¹ to less than or equal to about 3.54 E⁻⁴ S·cm⁻¹, and in certain aspects, optionally about 10⁻⁴ S·cm⁻¹. The “Young's modulus” is a mechanical property referring to a ratio of stress to strain for a given material. Young's modulus may be provided by the expression:

$$E = \frac{( stress )}{( strain )} = \frac{σ}{ϵ} = \frac{{L}_{o}}{Δ L} \times \frac{F}{A}$$

where engineering stress is σ, tensile strain is ϵ, E is the Young's modulus, L_{O} is an equilibrium length, ΔL is a length change under the applied stress, F is the force applied and A is the area over which the force is applied. “Soft” as used herein refers to materials having a Young's modulus of less than about 100 kPa.

The polyol preferably has a dielectric constant greater than or equal to about 31.7 to less than or equal to about 42. For example, in certain variations, the polyol may include glycerol, which has a dielectric constant greater than or equal to about 40 to less than or equal to about 42. The polyol may also include, in addition to or instead of the glycerol, other water-soluble polyol. In each instance, the organohydrogel may include greater than or equal to about 5 wt. % to less than or equal to about 60 wt. %, and in certain aspects, optionally greater than or equal to about 10 wt. % to less than or equal to about 40 wt. %, of the polyol.

The polyphenol may provide certain antibacterial properties to the organohydrogel without extensive cytotoxicity. For example, in certain variations, the polyphenol may include dopamine, tannic acid, and/or gallic acid. In further variations, the polyphenol may be selected from the group consisting of: dopamine, tannic acid, gallic acid, and combinations thereof. In each instance, the organohydrogel may include greater than or equal to about 0.01 g to less than or equal to about 0.1 g of the polyphenol for every gram of polymer (e.g., poly(vinyl alcohol)).

In accordance with various aspects of the present disclosure, the flexible, transparent organohydrogel may be used, for example, as a transparent, strain-sensing skin adhesive for motion monitoring. In accordance with other aspects of the present disclosure, the flexible, transparent organohydrogel may be used, for example, as a wound dressing. The water absorption and passthrough of the flexible, transparent organohydrogel makes the flexible, transparent organohydrogel particularly useful as a wound dress. Also, the transparent nature of the flexible, transparent organohydrogel may allow the wound to be observed without removal thereby reducing infection risks. In accordance with still other aspects of the present disclosure, the flexible, transparent organohydrogel may be used as a padding to provide moisture to dry skin and to ease itching conditions. For such skin-adhesive applications, ionic conductivity may not be necessary, allowing the composition to be modified accordingly. In accordance with still other aspects of the present disclosure, the flexible, transparent organohydrogel may be used as adhesive padding over healed wound and to prevent visible scar formation. In such instances, the three-dimensional network structure of the organohydrogel may help to prevent the aligned growth of skin tissue to quickly seal the wound. Quick wound sealing often lead to a morphology different from the normal skins having random distribution orientation of skin cells.

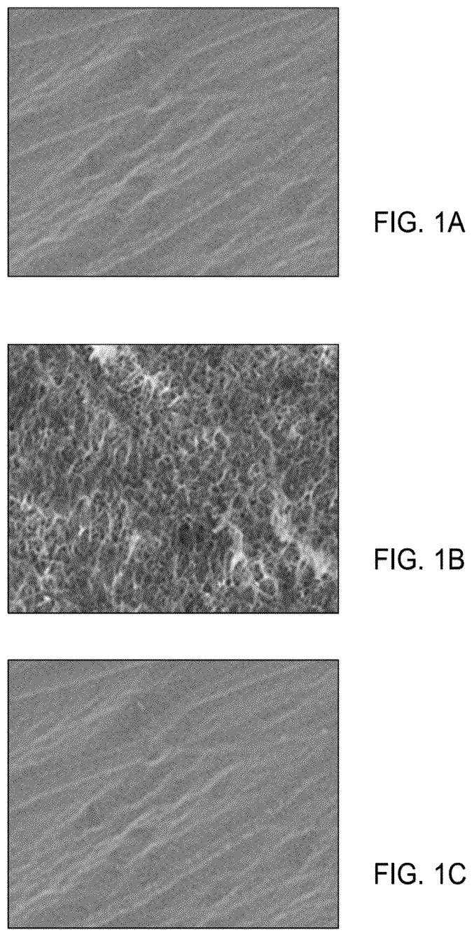

In various aspects, the present disclosure provides methods for preparing flexible, transparent organohydrogels, like those detailed above. As discussed above, poly(vinyl alcohol) networks formed by repeatedly freezing and thawing an aqueous poly(vinyl alcohol) solution are generally highly crystalline and therefore opaque, limiting their usefulness in applications where transparency is desired. Ion-conducting organohydrogels (e.g., poly(vinyl alcohol) gels) can be prepared by controlling crystallinity during the freezing-thawing process through fabrication in a binary water/polyol system, which can shrink and homogenize the porous structure of the organohydrogels, thereby increasing transparency. In the accordance with various aspects of the present disclosure, the crystallinity reduction of the organohydrogels (e.g., poly(vinyl alcohol) gels) can be achieved by interrupting poly(vinyl alcohol)-poly(vinyl alcohol) hydrogen bonding during ice templating through the addition and tuning of the polyol and the divalent metal salt. The polyol improves the mechanical, anti-freezing, and anti-drying properties of the organohydrogels. For example, incorporation of the metal and/or polyol at high loading levels may remove all of the porosity and also further densifies the organohydrogel. FIG. 1A is a microscopic image of an organohydrogel including a divalent metal salt and fabricated using a binary water/polyol system in accordance with various aspects of the present disclosure. By way of comparison, FIG. 1B is a microscopic image of an organohydrogel fabricated when the sol forms large ice crystals that push the polymer chains together, and FIG. 1C illustrate an organohydrogel fabricated with a divalent metal salt.

The strong hydrogen bonding of the polyol may allow the polyol to act as antifreeze by competing with water-water hydrogen bonding during ice formation, thereby preventing the formation of large ice crystals. While the formation of this scaly structure is beneficial for stretching enhancement due to the amorphousness, it needs to be balanced with the formation of strong hydrogen bonds, which can increase elastic modulus and reduce rupture strain. In the current instance, by disrupting the formation of light-scattering crystallites with the polyol and the divalent metal salt, the organohydrogel can be made transparent (e.g., % T>90%) while retaining sensing functionality.

Certain features of the current technology are further illustrated in the following non-limiting examples.

### EXAMPLES

The effects of polyol (e.g., glycerol) loading on tensile, adhesive, conductive, and resistivity strain-sensing properties of a poly(vinyl alcohol) organohydrogel fabricated in a binary glycerol/water system is explored using the examples summarized in Table 1 below. The samples were prepared by dissolving 1 gram of poly(vinyl alcohol) in glycerol/water solutions of ratios 0/100, 10/90, 15/85, 20/80, 25/75, or 40/60 wt. % to create 10 grams of a 10 wt. % poly(vinyl alcohol) solution in glycerol/water. Solutions were stirred vigorously for about 1 hour at about 80° C. then left to cool to room temperature. Upon cooling, about 2.03 grams of Zn(NO₃)₂*6H₂O was added and stirred at room temperature (e.g., greater than or equal to about 18° C. to less than or equal to about 24° C.) for about 1 hour. The solutions were then sonicated for about 5 minutes and left closed overnight to degas. Organohydrogels were then prepared by pouring about 6 grams of solution into a circular polystyrene mold measuring about 60 mm in diameter. The organohydrogels were then frozen for about 24 hours at about −20° C. and left to thaw for about 1 hour at about 20° C. This cycle was repeated 2 more times for the glycerol-free samples, then all gels were conditioned at about 20° C. and about 20% RH for about 18 hours.

TABLE 1
Organohydrogel Compositions and Molar Ratios

| Example | Description | nOH_{PVA}:nOH_{Gly}:nZn²⁺ |
|---|---|---|
| PVA | 10 wt. % PVA in water | 1:0:0 |
| GPZ00 | 10 wt. % PVA + Zn²⁺ in water | 1:0:0.3 |
| GPZ10 | 10 wt. % PVA + Zn²⁺ in Glycerol:Water 10:90 | 1:1.43:0.3 |
| GPZ15 | 10 wt. % PVA + Zn²⁺ in Glycerol:Water 15:85 | 1:2.145:0.3 |
| GPZ20 | 10 wt. % PVA + Zn²⁺ in Glycerol:Water 20:80 | 1:2.85:0.3 |
| GPZ25 | 10 wt. % PVA + Zn²⁺ in Glycerol:Water 25:75 | 1:3.61:0.3 |
| GPZ40 | 10 wt. % PVA + Zn²⁺ in Glycerol:Water 40:60 | 1:5.65:0.3 |

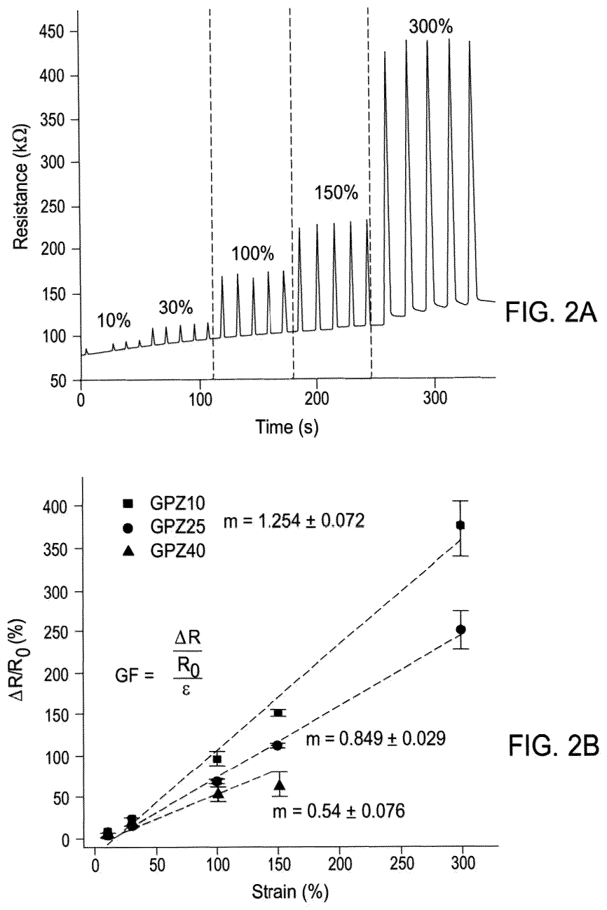

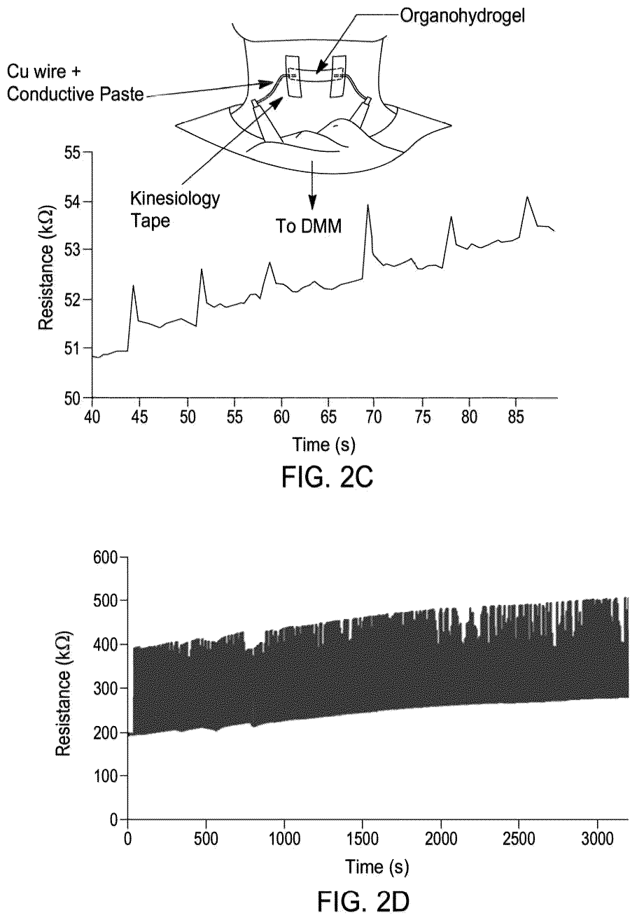

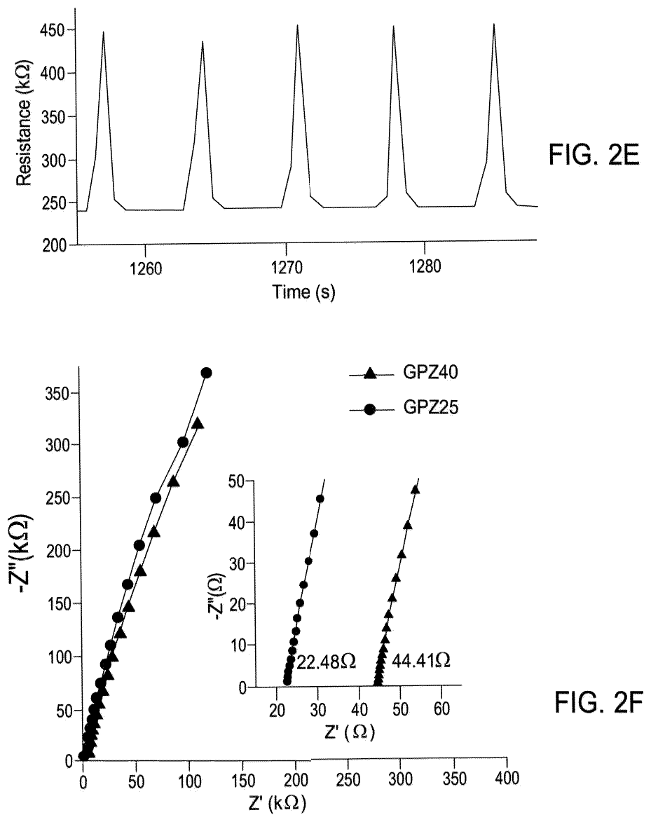

The GPZ gels demonstrate a mechano-responsive effect that operates at large stains, which is often desirable for sensors requiring not only sensitivity but also dynamic range. For example, FIG. 2A is a graphical demonstration illustrating an increase in resistance response to strains between 10% and 200% for the GPZ25 sample, where the x-axis represents time in seconds and the y-axis represents resistance (kΩ), and FIG. 2B is a graphical demonstration illustrating a precent resistance change (ΔR/R₀) plotted against the strain (%) where the slops represent a gauge factor (GF). As illustrated, the GPZ gels demonstrate strain sensitivity where increases in resistance are elicited by corresponding strains between 10% and 300%, and more specifically strain sensitivity decreases with increased glycerol content, indicating that high glycerol loading may increase resistance to ionic migration. By way of example, FIG. 2C is a graphical demonstration illustrating the resistance response to strains for the GPZ25 sample as placed over the hyoid cartilage of a human subject and after a repeated response (i.e., “Hello”) was elicited, where the x-axis represents time in seconds and the y-axis represents resistance (kΩ). FIG. 2D is a graphical demonstration illustrating the relatively consistent response over 1000 pulls of the GPZ25 sample as placed over the hyoid cartilage of a human subject, where the x-axis represents time in seconds and the y-axis represents resistance (kΩ). FIG. 2E is a graphical demonstration providing an exploded view of the relatively consistent response of the GPZ25 sample as placed over the hyoid cartilage of a human subject, where the x-axis represents time in seconds and the y-axis represents resistance (kΩ).

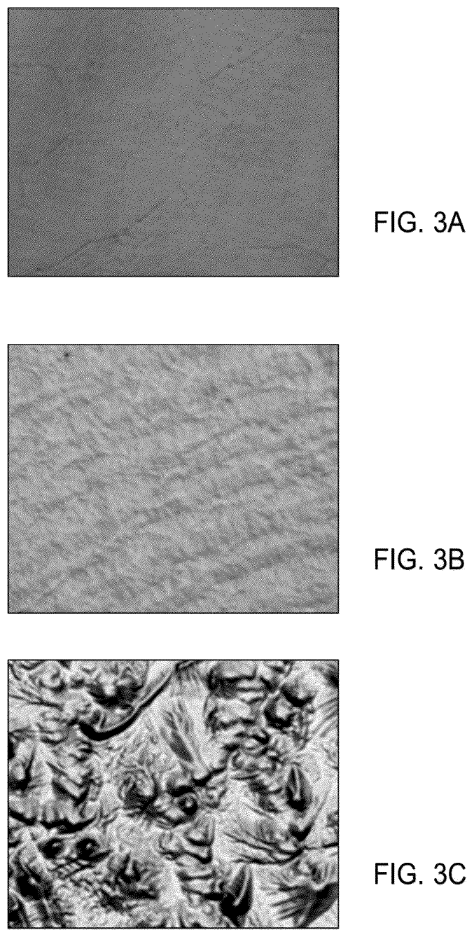

Electrical Impedance Spectroscopy (EIS) may be used to determine ionic conductivity. For example, the conductivities of GPZ25 and GPZ40 samples as characterized by EIS is illustrated in FIG. 2F. The GPZ25 sample has an ionic conductivity of 3.54 E⁻⁴ S·cm⁻¹, while the GPZ40 sample has an ionic conductivity of 1.79 E⁻⁴ S·cm⁻¹, showing a decrease in ionic conductivity as more glycerol is added and polymer chain mobility is restricted. The conductivity of the GPZ samples is due to ion percolation through amorphous regions and also to diffusion of the liquid-phase electrolyte through the network. As both pathways involve ion motion through the network, decreases in polymer mobility can impede ion mobility, thereby affecting response sensitivity. Furthermore, the amount of glycerol can affect transparency and wrinkling of the organohydrogel. Increasing amounts of glycerol can increase wrinkling, gel shrinkage during fabrication, and haze due to light-scattering wrinkles on the surface of the organohydrogel. For example, by way of comparison, FIG. 3A is a microscopic image of a surface of the GPZ10 sample, FIG. 3B is a microscopic image of a surface of the GPZ25 sample, and FIG. 3C is a microscopic image of a surface of the GPZ40 sample. Achieving a preferred organohydrogel therefore requires balance between desirable mechanical stability and polymer/ion mobility, which can be achieved by tuning the amount of glycerol in this system.

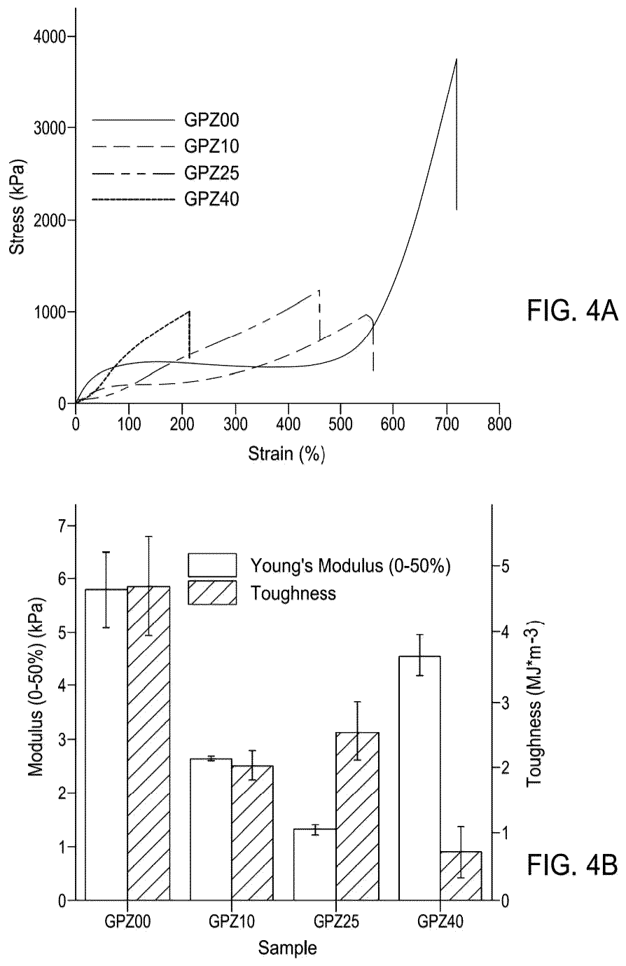

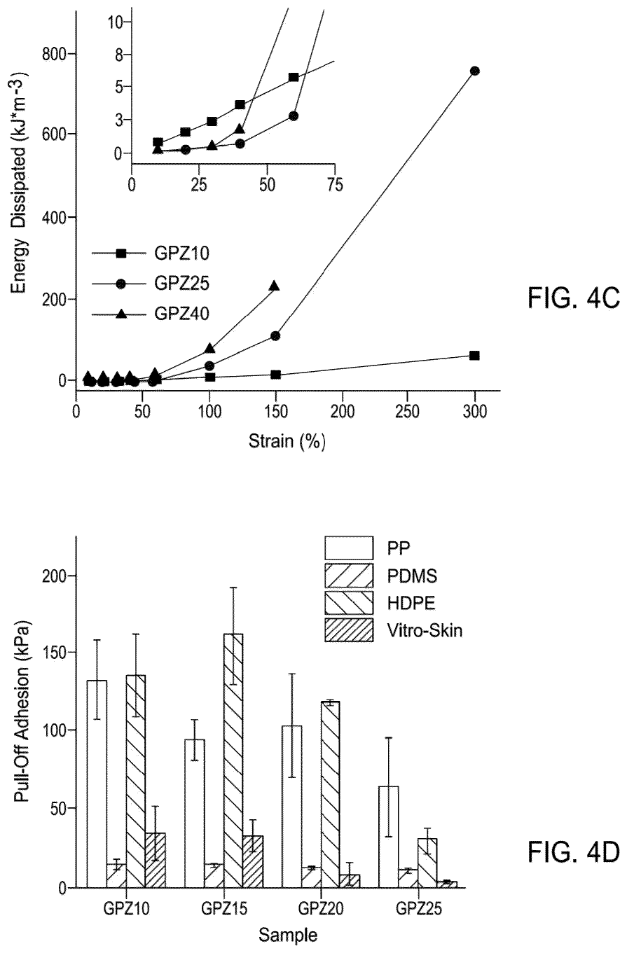

The inclusion of the divalent metal salt (e.g., Zn(NO₃)₂) may disrupt the formation of crystallites in the organohydrogel and results in a transparent, amorphous gel with reduced crystallinity. For example, as illustrated in FIG. 4A, under tensile strain, the GPZ00 sample may have a short elastic response regime, long plastic deformation regime, and may undergo strain hardening before fracture. FIG. 4A is a graphical demonstration comparing the stress-stain curves for the GPZ00 sample, the GPZ10 sample, the GPZ25 sample, and the GPZ40 sample, where the x-axis represents strain (%) and the y-axis represents stress (kPa). The plastic deformation occurs as the loosely associated poly(vinyl alcohol) chains are allowed to slide against each other, and irreversible strain hardening occurs as the stretched chains realign and recrystallize along the tensile axis. As glycerol content is increased, the organohydrogel may behave more like an elastic material, developing a more linear stress-strain response, as illustrated. The reduced elongation and elastic behavior are likely due to increased hydrogen bonding within the material by glycerol, which prevents the chain slippage that occurs in the plastic deformation regime of the GPZ00 sample. For example, FIG. 4B is a graphical demonstration comparing the Young's Modulus and toughness for the GPZ00 sample, the GPZ10 sample, the GPZ25 sample, and the GPZ40 sample. As illustrated, there is a decrease in Young's Modulus from 0-50% strain as glycerol content is increased from 0:100 to 10:90 glycerol:water, after which the plasticization effect by the glycerol is overtaken by the high amount of hydrogen bonding between poly(vinyl alcohol) chains and glycerol.

The baseline increases across repeated 300% pulls shown in FIG. 2A is likely due to the hysteresis at high strain caused by energy dissipation through hydrogen bond breaking, however, strains up to 150% do not exhibit this behavior. Most mechano-sensing applications take place at relatively small strains, however, strains up to 300% were investigated. FIG. 4C is a graphical demonstration illustrating the energy dissipation for the GPZ10 sample, the GPZ25 sample, and the GPZ40 sample, where the x-axis is strain (%) and the y-axis is energy dissipated (kJ*m⁻³). As illustrated, a significant amount of hysteresis is observed at strains greater than 60% for the GPZ25 and the GPZ40 samples. At these strains, the amount of energy dissipated with each subsequent strain cycle may increase with the amount of glycerol added due to increased abundance of hydrogen bonds to rupture. The effect of glycerol on hysteresis is more pronounced below 60% strain. Increased synergistic hydrogen bonding within the material delays the onset of hysteresis for the GPZ25 and the GPZ40 samples, indicating that the plasticization effect may be canceled out by the increased hydrogen bonding at these loading levels. Conversely, the GPZ10 sample may experience significant hysteresis at very small strains, as this amount of glycerol does not provide sufficient hydrogen bonding to overcome the interrupted poly(vinyl alcohol)-poly(vinyl alcohol) interactions.

As illustrated, strain sensitivity and recoverability are in opposition. To have a wide dynamic range, fast recovery after deformation is required, therefore the material should resist hysteresis, but it is unavoidable for non-covalently crosslinked networks with large dynamic range. Conversely, a lower Young's Modulus may be beneficial for strain sensitivity. The GPZ10 sample has the highest sensitivity of the tested samples but the lowest strain recovery ability (Supplemental Information, Figure S4). Thus, the GPZ25 sample may be a more suitable strain sensor balancing sensitivity and recovery. For the GPZ25 sample, the usable elastic deformation region appears to be from 0-150% strain.

Low glycerol-content organohydrogels may adhere to skin due to their softness. In certain variations, hydrogen bonding between poly(vinyl alcohol) and skin surface amines may improve adhesion. The pull-off adhesion to LSE substrates may be studied to predict glycerol loading effects on skin adhesion. Increasing glycerol content from about 10% to about 25% shows a reduction in adhesion to high-density polyethylene (HDPE) and polypropylene (PP), likely as a result of a reduction in the amount of surface poly(vinyl alcohol) hydroxyls and reduced wetting-out capability of the higher-modulus gel. For example, FIG. 4D is a graphical demonstration illustrating the effects of substrate surface energy on organohydrogel adhesion using the pull-off adhesion of the example organohydrogels to several low surface energy substrates, including high-density polyethylene (HDPE), polypropylene (PP), silicone, and VITRO-SKIN® with N19 topography. As illustrated, optimal adhesive strength appears to be between about 10 wt. % and about 15 wt. % glycerol. This optimal adhesion strength is likely related to the ability of the organohydrogel to form good contact with the surface, since the low-glycerol gels are softer. The elastic response may be required for recoverability opposes the softness required for adhesion as well.

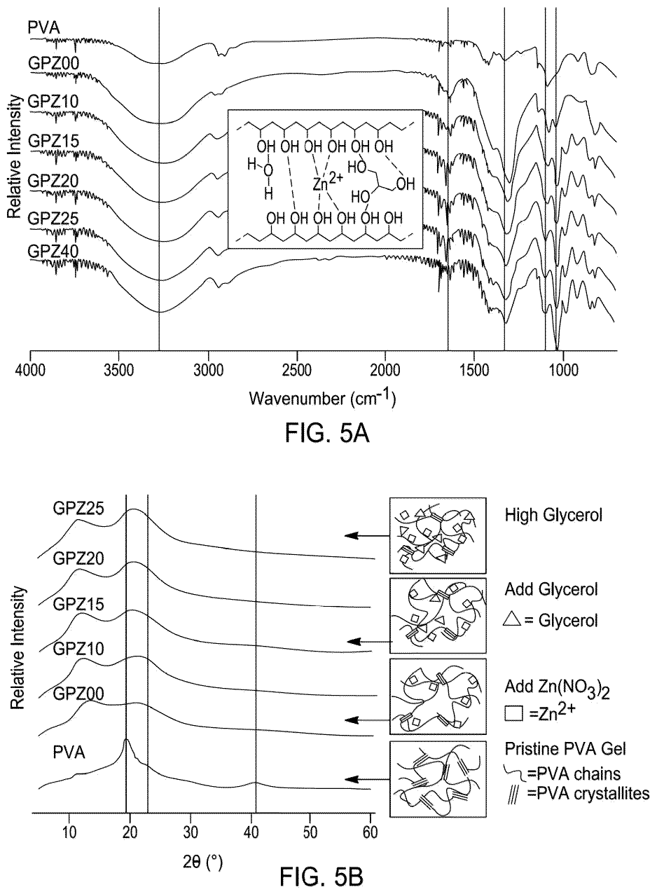

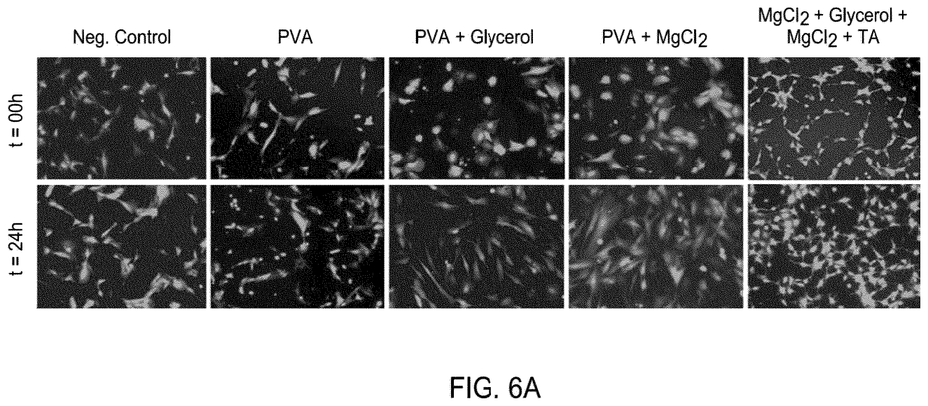

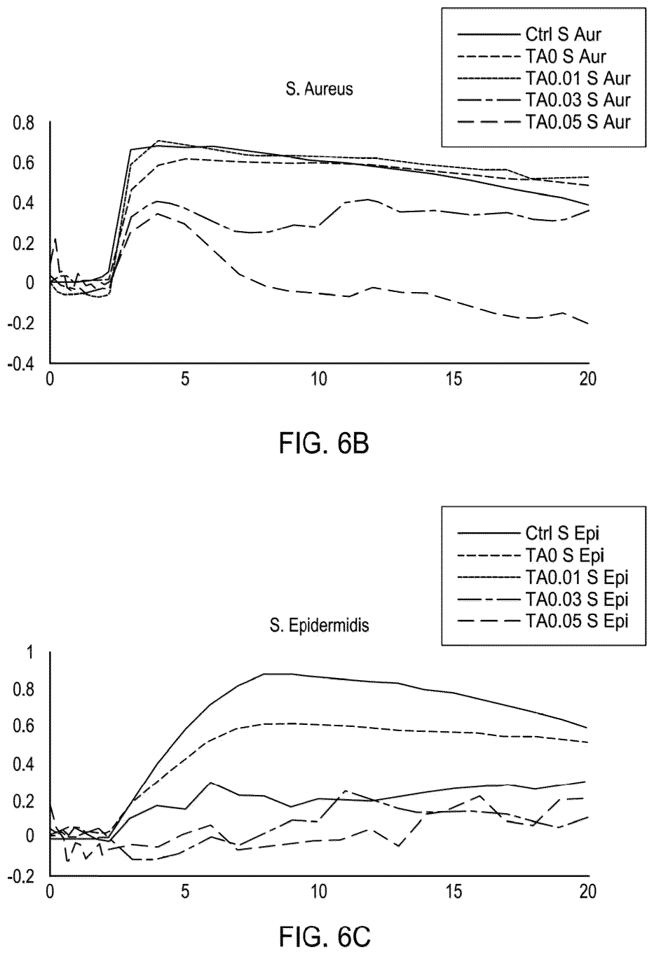

FIG. 5A is a graphical demonstration illustrating the Fourier-transform infrared spectroscopy (FTIR) spectra of example flexible, transparent organohydrogels, where the x-axis represents wavenumber (cm⁻¹), and the y-axis represents relative intensity. In the FTIR spectrum of poly(vinyl alcohol) the band at 3271 cm⁻¹ corresponds to an inter molecular O—H stretch, while the low glycerol loading example (e.g., the GPZ10 sample) lowers the O—H stretch peak to 3245 cm⁻¹, which indicates the formation of hydrogen-bonds between glycerol and poly(vinyl alcohol). Table 1 above lists the molar ratios of poly(vinyl alcohol) hydroxyls to glycerol hydroxyls ((nOH_{PVA}:nOH_{Glycerol}). The GPZ10 sample may have has a nOH_{PVA}:nOH_{Glycerol} of about 1:1.43, which indicates that the moles of poly(vinyl alcohol)-OH are about equal to the moles of glycerol-OH. As more glycerol is introduced and the nOH_{PVA}:nOH_{Glycerol} ratio increases to about 1:5.65, the band may shift, as illustrated, to a higher wavenumber closer to that of glycerol, which indicates that more poly(vinyl alcohol)-poly(vinyl alcohol) bonds are broken as poly(vinyl alcohol)-glycerol interactions are formed, shifting the peak towards the O—H stretch value for pure glycerol. The O—H stretch value for pure glycerol is higher than that for poly(vinyl alcohol). Similar behavior is observed in for the band at about 1100 cm⁻¹ corresponding to the C—C—O stretch of a secondary alcohol, which blue-shifts with increased glycerol loading as poly(vinyl alcohol)-poly(vinyl alcohol) hydrogen bonds are switched to PVA-glycerol hydrogen bonds.

In pristine poly(vinyl alcohol), there are very few primary alcohols (occupying hydrolyzed chain ends) compared to secondary alcohols, whereas the majority of alcohol groups in glycerol are primary alcohols. Changes to the primary alcohol C—C—O peak allow hydrogen bonding within the organohydrogel as glycerol loading is increased to be monitored. The primary alcohol C—C—O stretch is a shoulder peak in the IR spectrum of poly(vinyl alcohol) and full peak in the IR spectrum of the GPZ00 sample at 1042 cm⁻¹. This is likely due to divalent metal salt (e.g., Zn(NO₃)₂) interrupting the poly(vinyl alcohol)-poly(vinyl alcohol) interactions, liberating chain end defects that plasticize the gel as shown in the stress-strain curve. After adding glycerol, this peak may red-shift to about 1038 cm⁻¹, indicating that the primary alcohols in glycerol are interacting the with alcohol groups of poly(vinyl alcohol). Increased glycerol may cause the primary alcohol C—C—O band to become more pronounced, owing to the increased presence of primary alcohol groups. The peak may shift further to about 1036 cm⁻¹, indicating that these primary alcohol groups are participating in hydrogen bonding.

The x-ray diffraction (XRD) spectrum for poly(vinyl alcohol) includes three distinct peaks: a crystalline peak around 2θ=18-21° corresponding to the [101̅] lattice direction, an amorphous peak around 2θ=23°, and a compound peak around 2θ=41°. In addition, residual water may have a peak around 2θ=30°. FIG. 5B is a graphical demonstration illustrating the effects of including a divalent metal salt (e.g., Zn(NO₃)₂) and polyol (e.g., glycerol) in the organohydrogel, where the x-axis represents 2θ and the y-axis represents relative intensity. As illustrated, the organohydrogel drastically broadens the crystalline peak, which denotes a reduction in crystallite size. Table 3 below summarizes the crystallite dimensions of each sample, where the poly(vinyl alcohol) crystallites are roughly about 5.93 nanometers (nm). The crystallite dimensions decrease with the addition of the divalent metal salt and the polyol but begin to increase as more of the polyol is added. As illustrated, as more glycerol is added, the intensity of the crystalline peak increases. At high loading levels of glycerol, increase in the peak at 2θ is about 20°, which is likely due to stronger poly(vinyl alcohol)-glycerol interactions correlating to the decrease in elongation and increase in modulus.

TABLE 2
Crystalline properties of poly(vinyl alcohol)
and sample organohydrogels calculated from XRD.

| Sample | 2 θ [°] | d-spacing [nm] | Apparent crystallite dimensions [nm] |
|---|---|---|---|
| PVA | 19.459 | 0.46 | 5.93 |
| GPZ00 | 17.03 | 0.52 | 2.57 |
| GPZ10 | 20.55 | 0.43 | 1.36 |
| GPZ15 | 20.64 | 0.42 | 1.57 |
| GPZ20 | 21.08 | 0.43 | 2.28 |
| GPZ25 | 20.73 | 0.43 | 2.29 |

The transport of ions through the sample organohydrogels requires high polymer segmental mobility, immobilizing the polymer through a high crosslink density or strong intermolecular interactions may reduce ion conductivity by increasing resistance to ionic migration. Native poly(vinyl alcohol) organohydrogels are physically crosslinked through the formation of crystalline domains that can be observed using XRD. Disruption of the poly(vinyl alcohol)-poly(vinyl alcohol) interactions upon addition of ions may prevent the formation of significantly large crystallites, giving an amorphous structure that aids in ionic conduction. As illustrated in the inset of FIG. 5B, glycerol may affect ion mobility. For example, glycerol may promote intermolecular interactions and may act as a physical crosslinker for poly(vinyl alcohol) without significantly increasing crystallinity. However, at high glycerol loadings, poly(vinyl alcohol)-glycerol and glycerol-glycerol interactions may dominate, inducing shrinkage, wrinkling, stiffness, and inhomogeneity that may eventually degrade the mechanical properties and also the ionic conduction of the organohydrogel. At glycerol loadings where moles of glycerol —OH far exceeds moles of poly(vinyl alcohol)-OH, only one or two —OH on each glycerol molecule can participate in hydrogen-bonding with poly(vinyl alcohol), thus a critical point of crosslinking may be reached and there may be some free glycerol molecules present, which introduces inhomogeneity and may degrade sensor properties. Through careful modulation of glycerol content, the mechanical properties of this organohydrogel can be tuned while maintaining amorphousness and transparency, which is crucial for the engineering of strain sensors.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A flexible, transparent organohydrogel for skin-based applications comprising:
a polymeric matrix comprising crosslinked hydrophilic polymers;
a divalent metal salt configured to disrupt crystalline interactions of the polymeric matrix; and
a polyol configured to reduce a strength of Coulomb attraction among cations and anions of the divalent metal salt and among polymer chains of the polymeric matrix thereby increasing the flexibility of the organohydrogel.

2. The flexible, transparent organohydrogel of claim 1, wherein the divalent metal salt is selected from the group consisting of: magnesium chloride (MgCl₂), zinc chloride (ZnCl₂), zinc nitrate (Zn(NO₃)₂), magnesium nitrate (Mg(NO₃)₂) and calcium chloride (CaCl₂)), and combinations thereof.

3. The flexible, transparent organohydrogel of claim 1, wherein the polymeric matrix comprises poly(vinyl alcohol) (PVA).

4. The flexible, transparent organohydrogel of claim 3, wherein the divalent metal salt disrupts the crystalline interactions of the polymer matrix as represented by nOH(PVA):nM²⁺ that occurs in an amount greater than or equal to about 10:1 to less than or equal to about 10:3, where M²⁺ represents the metal group of the divalent metal salt.

5. The flexible, transparent organohydrogel of claim 1, wherein the polyol has a dielectric constant greater than or equal to about 31.7 to less than or equal to about 42.

6. The flexible, transparent organohydrogel of claim 1, wherein the flexible, transparent organohydrogel includes greater than or equal to about 5 wt. % to less than or equal to about 60 wt. % of the polyol.

7. The flexible, transparent organohydrogel of claim 1, wherein the polyol comprises a water-soluble polyol.

8. The flexible, transparent organohydrogel of claim 1, wherein the polyol comprises glycerol.

9. The flexible, transparent organohydrogel of claim 1, wherein the flexible, transparent organohydrogel has a gauge factor (GF) greater than or equal to about 0.54 to less than or equal to about 1.254.

10. The flexible, transparent organohydrogel of claim 1, wherein the flexible, transparent organohydrogel has a dynamic range greater than or equal to about 5% to less than or equal to about 300%.

11. The flexible, transparent organohydrogel of claim 1, wherein the flexible, transparent organohydrogel has a Young's modulus greater than or equal to about 0.1 kPa to less than or equal to about 7 kPa.

12. The flexible, transparent organohydrogel of claim 1, wherein the flexible, transparent organohydrogel has an ionic conductivity greater than or equal to about 1.79 E⁻⁴ S·cm⁻¹ to less than or equal to about 3.54 E⁻⁴ S·cm⁻¹.

13. The flexible, transparent organohydrogel of claim 1, further comprising:
a polyphenol.

14. The flexible, transparent organohydrogel of claim 13, wherein the polyphenol is an antibacterial material.

15. The flexible, transparent organohydrogel of claim 13, wherein the polyphenol is selected from the group consisting of: dopamine, tannic acid, gallic acid, and combinations thereof.

16. The flexible, transparent organohydrogel of claim 13, wherein the flexible, transparent organohydrogel comprises greater than or equal to about 0.01 g to less than or equal to about 0.1 g of the polyphenol for every gram of the crosslinked hydrophilic polymers in the polymeric matrix.

17. The flexible, transparent organohydrogel of claim 1, wherein the polymeric matrix further comprises hydrophobic molecules configured to improve adhesion for the skin-based applications.

18. The flexible, transparent organohydrogel of claim 17, wherein the hydrophobic molecules comprise a propyl group.

19. A flexible, transparent organohydrogel for skin-based applications comprising:
a polymeric matrix comprising poly(vinyl alcohol) (PVA);
a divalent metal salt configured to disrupt crystalline interactions of the polymeric matrix, the divalent metal salt selected from the group consisting of: magnesium chloride (MgCl₂), zinc chloride (ZnCl₂), zinc nitrate (Zn(NO₃)₂), magnesium nitrate (Mg(NO₃)₂) and calcium chloride (CaCl₂)), and combinations thereof, wherein a ratio of hydroxyl (—OH) groups in the poly(vinyl alcohol) to a metal group of the divalent metal salt is represented by nOH(PVA):nM²⁺ and is greater than or equal to about 10:1 to less than or equal to about 10:3; and
greater than or equal to about 5 wt. % to less than or equal to about 60 wt. % of glycerol configured to reduce a strength of Coulomb attraction among cations and anions of the divalent metal salts and among polymer chains of the polymeric matrix thereby increasing the flexibility of the organohydrogel.

20. The flexible, transparent organohydrogel of claim 19, wherein the flexible, transparent organohydrogel further comprises at least one of:
a polyphenol having antibacterial properties; and
a propyl group selected to improve adhesion for the skin-based applications.
